(19) United States
(12) Patent Application Publication
(10) Pub. No.: US 2013/0224303 A1
(43) Pub. Date: Aug. 29, 2013

(54) PROCESS OF PRODUCING SHELF STABLE PROBIOTIC FOOD

(71) Applicants: Arup Nag, Palmerston North (NZ); Shantanu Das, Palmerston North (NZ); Harjinder Singh, Palmerston North (NZ)

(72) Inventors: Arup Nag, Palmerston North (NZ); Shantanu Das, Palmerston North (NZ); Harjinder Singh, Palmerston North (NZ)

(73) Assignee: MASSEY UNIVERSITY, Palmerston North (NZ)

(21) Appl. No.: 13/819,282

(22) PCT Filed: Aug. 25, 2011
(86) PCT No.: PCT/NZ2011/000169

§ 371 Date: May 10, 2013

(30) Foreign Application Priority Data
Aug. 26, 2010 (AU) 2010903824
May 9, 2011 (NZ) 592728

(51) Int. Cl. A23L 1/30; A61K 35/74; A61K 9/14

(52) U.S. Cl. CPC A23L 1/3014; A61K 9/14; A61K 35/747; A61K 35/745
USPC 424/490; 426/61; 424/93.45; 424/93.4

(57) ABSTRACT

The invention provides an ingredient containing shelf-stable probiotic microorganisms for use in preparing probiotic food products requiring long shelf-life. The ingredient is prepared by coating particles or an agglomeration thereof with a suspension of osmotically shocked probiotic microorganisms and drying the product.

### FIELD OF THE INVENTION

The present invention relates generally to a process for producing shelf stable foods containing viable probiotic microorganisms.

### BACKGROUND

Probiotic microorganisms (probiotics) are microorganisms that beneficially improve host health by increasing beneficial bacteria in the human gut. It is believed that probiotics improve health by suppressing pathogenic bacteria, preventing intestinal diseases, improving lactose utilization in lactose intolerant people and helping to control serum cholesterol levels (Adamiec, 2009).

Probiotic microorganisms (also referred to as probiotic cells) are commonly incorporated in dairy products that have relatively short shelf-life and need to be kept chilled. For example, currently probiotics are often added to fermented milks and yoghurts.

There is however, an increasing interest in the delivery of probiotics in dry or intermediate moisture shelf stable foods.

Dry food products usually have a shelf life at room temperature of between six months to one year. However, under these conditions probiotic bacteria generally lose their viability within a few months (Ubbink & Kruger, 2006) and decrease to a functionally insignificant level within a few weeks.

It has also been difficult to maintain the viability of probiotics in intermediate moisture foods when stored under ambient conditions (Lee & Salminen, 2009).

The survival of probiotics in shelf-stable food has high commercial importance. Researchers have therefore attempted to encapsulate probiotic bacteria using various different microencapsulation techniques and a wide range of food ingredients in an attempt to retain their viability during product processing, storage and gastric transit (Anal and Singh, 2007). Various methods of drying have also been used.

However, these techniques have given varying results and no process has been developed that can retain the viability of probiotics at room temperature for a period of time long enough to be commercially useful in shelf stable products.

There is therefore a need in the art to provide a process for obtaining a product that retains the viability of probiotic microorganisms at room temperature, or at least provides the public with a useful choice.

### SUMMARY OF THE INVENTION

The invention relates to a process for producing shelf stable viable probiotic microorganisms for use directly as food or as an ingredient in food.

In a first aspect, the present invention provides a process for preparing consumable coated particles or an agglomeration thereof comprising:
a) at least partially coating particles comprising carbohydrate, protein and optionally fat with a suspension of osmotically shocked probiotic microorganisms; and
b) drying the coated particles or agglomeration thereof;
   wherein the consumable coated particles or agglomeration thereof include viable probiotic microorganisms.

In a second aspect, the present invention provides a process for preparing consumable coated particles or an agglomeration thereof comprising:
a) at least partially coating particles comprising carbohydrate, protein and optionally fat with a suspension of osmotically shocked probiotic microorganisms; and
b) drying the coated particles or agglomeration thereof;
   wherein the consumable coated particles or agglomeration thereof include viable probiotic microorganisms, and wherein steps a) and b) are carried out using a fluidised bed apparatus.

In a third aspect, the invention provides a process for preparing consumable coated particles or an agglomeration thereof comprising:

a) at least partially coating particles comprising carbohydrate, protein and optionally fat with a suspension of osmotically shocked probiotic microorganisms; and
b) drying the coated particles or agglomeration thereof and;
wherein the dried coated particles or agglomeration thereof include at least 10⁷ cfu/gram of viable probiotic microorganisms; and
wherein the concentration of viable probiotic microorganisms decreases less than 2 log cfu/gram after 6 months storage at room temperature.

In one embodiment the agglomeration of dried coated particles includes embedded probiotic microorganisms.

In a fourth aspect, the invention provides consumable coated particles or an agglomeration thereof produced by the process of the invention.

In a fifth aspect, the invention provides consumable particles comprising carbohydrate, protein and optionally fat, coated with osmotically shocked probiotic microorganisms; and/or an agglomeration of said particles.

In a sixth aspect, the invention provides a consumable product comprising consumable coated particles or agglomeration thereof according to the invention.

In a seventh aspect, the invention provides a pharmaceutical composition comprising consumable coated particles or an agglomeration thereof according to the invention, and a pharmaceutically acceptable excipient.

In the above aspects:

In one embodiment the bacteria are selected from the group comprising L. casei CRL431 (ATCC accession number 55344), L. casei ATCC 393, L. acidophilus ATCC 4356, L. rhamnosus ATCC 53103 and B. lactic BB12.

In another embodiment, the particles comprising carbohydrate, protein and optionally fat comprise dairy powder, preferably milk powder.

In another embodiment, the coated particles or agglomeration thereof are dried at 20-50° C.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### BRIEF DESCRIPTION OF THE DRAWINGS

FIG. 1 is a diagram showing a schematic process for a top spray fluid bed agglomeration drying system that may be useful in the present invention.

FIG. 2 is a graph showing the growth curve of L. casei CRL431 cells in MRS broth. The values posted on the graph are mean OD values of 3 samples at each time point.

FIG. 3 is a graph showing the stability of embedded L. casei CRL431 cells stored at 25° C. for 48 weeks when the cells were pre-adapted with heat (), osmotic (♦), combined stress (x) and control with no stress (▴), compared with freeze dried L. casei CRL431 culture (▪).

FIG. 4 is a graph showing the stability of probiotic bacteria L. casei CRL431 when embedded into a composite matrix and fortified in cereal bars (▪) and chocolate spreads (▴).

FIG. 5 is a graph showing the cell loading of alginate capsules before and after controlled drying. Initial cell counts in all four wet samples were above 10.00 log cfu/gm.

FIG. 6 is a graph showing L. casei CRL431 viability when encapsulated in alginate and stored at 25° C. for 4 weeks.

FIG. 7 is a graph showing L. casei CRL431 viability when encapsulated in alginate and further coated in lipid and stored at 25° C. for 4 weeks.

FIG. 8 is a series of graphs showing cell viability after storage at 25° C. for up to 8 months. Osmotically stressed cells (▪) were compared to unstressed cells (♦) of L. acidophilus ATCC 4356 (FIG. 8A), L. rhamnosus ATCC 53103 (FIG. 8B), L. casei ATCC 393 (FIG. 8C) and Bifidobacterium lactic BB 12 (FIG. 8D). Freeze dried cells are shown by (▴).

FIG. 9 is a graph showing the stability of embedded L. casei CRL431 stored at 25° C. for 12 weeks. The graph shows the viability of L. casei cells osmotically stressed and encapsulated with the novel technology (♦) in comparison to osmotically stressed cells dried using spray drying (x) and freeze drying ( ) Control sample () represents commercially available freeze dried granules of the same bacteria.

FIG. 10 is a graph showing the particle size distributions of the probiotic ingredient of the invention (♦) in comparison with whole milk (▴) and skim milk (▪) powders.

FIG. 11 is a graph showing showing the flow properties of the probiotic ingredient of the invention (▪) in comparison with whole milk (▴) and skim milk (♦) powders.

FIG. 12 is a graph showing the sensory qualities of malted milk beverage with and without addition of the probiotic ingredient of the invention.

FIG. 13 is a graph showing the storage viability of products of the invention (♦) compared to osmotically stressed L. casei cells encapsulated in milk protein isolate (▪) and osmotically stressed L. casei cells encapsulated in glucose (▴).

### DETAILED DESCRIPTION OF THE INVENTION

The process of the invention can be used to prepare consumable particles and agglomerations thereof that includes viable probiotic microorganisms. These consumable particles and agglomerations can be used to prepare consumable products, for example, foods, beverages, dietary supplements and nutraceuticals.

The microorganisms remain viable in the consumable product for a relatively long period—making the consumable product “shelf-stable” with respect to the probiotic microorganisms.

The probiotic microorganisms remain viable because they are incorporated into and onto the coated particles or agglomerations of particles comprising carbohydrate, protein and optionally fat during the process of the invention.

The process of the invention therefore, produces consumable particles that can be used as ingredients in the production of probiotic-containing consumable food products that do not need to be refrigerated to maintain the viability of the probiotic microorganisms.

### DEFINITIONS

The term “comprising” as used in this specification means “consisting at least in part of”. When interpreting statements in this specification which include that term, the features, prefaced by that term in each statement or claim, all need to be present but other features can also be present. Related terms such as “comprise” and “comprised” are to be interpreted in the same manner.

The term “coated particles” includes particles that have been partially coated but does not include particles that form part of a liquid suspension. In other words the particles provide shape to the resulting product.

The term “at least partially coating” means that the particles to be coated have contact with some bacterial suspension. The particles might be, but do not need to be, fully covered with bacterial suspension.

The term “consumable product” as used in this specification means a product that is safe to be consumed by humans and/or animals. It may be a food or a product that is mixed with water, milk, juice or any other liquid to become a beverage. It may also be a dietary supplement or nutraceutical.

### Probiotic Microorganisms for Use in the Process of the Invention

The Food and Agriculture Organization (FAO) of the United Nations and the World Health Organization (WHO) define probiotic microorganisms as “live microorganisms which when administered in adequate amounts confer a health benefit for the host”. Products containing at least 10⁶-10⁷ cfu/gram are considered to provide a health benefit. (FAO/WHO, 2001).

Probiotics useful in the invention therefore include any microorganism that has been determined to have a beneficial effect on the host and may include strains that have not yet been identified as having a beneficial host effect.

The consumable coated particles or agglomeration thereof may include probiotic microorganisms from one or more strains, species or genera.

Bacteria such as lactic acid bacteria and bifidobacteria are known to be useful as probiotics but other microorganisms such as certain types of yeasts may also be useful in the process of the invention.

For example, probiotic strains useful in the present invention may be selected from the group comprising: yeasts such as Saccharomyces, Debaromyces, Candidaw Pichia and Torulopsis; moulds such as Aspergillus, Rhizopus, Mucor, Penicillium and Torulopsis; and bacteria such as the genera Bifidobacterium, Bacteroides, Clostridium, Fusobacterium, Melissococcus, Propionibacterium, Streptococcus, Enterococcus, Lactococcus, Kocuriaw, Staphylococcus, Peptostrepococcus, Bacillus, Pediococcus, Micrococcus, Leuconostoc, Weissella, Aerococcus, Oenococcus and Lactobacillus or a mixture thereof.

In one embodiment, the probiotic microorganism for use in the invention is selected from the group comprising: Aspergillus niger, A. oryzae, Bacillus coagulans, B. lentus, B. licheniformis, B. mesentericus, B. pumilus, B. subtilis, B. natto, Bacteroides amylophilus, Bac. capillosus, Bac. ruminocola, Bac. suis, Bifidobacterium adolescentis, B. animalis, B. breve, B. bifidum, B. infantis, B. lactis, B. longum, B. pseudolongum, B. thermophilum, Candida pintolepesii, Clostridium butyricum, Enterococcus cremoris, E. diacetylactis, E. faecium, E. intermedius, E. lactis, E. muntdi, E. thermophilus, Escherichia coli, Kluyveromyces fragilis, Lactobacillus acidophilus, L. alimentarius, L. amylovorus, L. crispatus, L. brevis, L. casei, L. curvatus, L. cellobiosus, L. delbrueckii ss. bulgaricus, L. farciminis, L. fermentum, L. gasseri, L. GG, L. helveticus, lactis, L. plantarum, L. bulgaricus, L. johnsonii, L. reuteri, L. rhamnosus, L. sakei, L. salivarius, Lactococcus lactis, Leuconostoc mesenteroides, P. cereviseae (damnosus), Pediococcus acidilactici, P. pentosaceus, Propionibacterium freudenreichii, Prop. shermanii, Saccharomyces cereviseae, Staphylococcus carnosus, Staph. xylosus, Streptococcus infantarius, Strep. salivarius ss. thermophilus, Strep. thermophilus, Strep. lactis and mixtures thereof.

In another embodiment, the probiotic microorganism is selected from the group comprising: Bifidobacterium breve R070, Bifidobacterium breve strain yakult, Bifidobacterium lactis Bb12, Bifidobacterium longum R023, Bifidobacterium bifidum R071, Bifidobacterium infantis R033, Bifidobacterium longum BB536, Bifidobacterium lactis BB12, Bifidobacterium lactis HN019 (HOWRU), Bifidobacterium longum SBT-2928 and mixtures thereof.

In another embodiment, the probiotic microorganism is selected from the group comprising: Lactobacillus plantarum 299v, Lactobacillus acidophilus BG2FO4, Lactobacillus acidophilus INT-9, Lactobacillus plantarum ST31, Lactobacillus reuteri, Lactobacillus johnsonii LA1, Lactobacillus acidophilus NCFB 1748, Lactobacillus casei Shirota, Lactobacillus acidophilus NCFM, Lactobacillus acidophilus DDS-1, Lactobacillus delbrueckii subspecies delbrueckii, Lactobacillus delbrueckii subspecies bulgaricus type 2038, Lactobacillus acidophilus SBT-2062, Lactobacillus brevis, Lactobacillus salivarius UCC 118, Lactobacillus casei 431, Lactobacillus casei ATCC 393, Lactobacillus acidophilus ATCC 4356, Lactobacillus rhamnosus ATCC 53103, Lactobacillus plantarum ATCC 8014, Lactobacillus plantarum LP293V, Lactobacillus paracasei subsp paracasei F19 and mixtures thereof.

In another embodiment, the probiotic microorganism is selected from the group comprising: Lactococcus lactis subspecies cremoris (Streptococcus cremoris), Lactococcus lactis subspecies lactis NCDO 712, Lactococcus lactis subspecies lactis NIAI 527, Lactococcus lactis subspecies lactis NIAI 1061, Lactococcus lactis subspecies lactis biovar diacetylactis NIAI 8W, Lactococcus lactis subspecies lactis biovar diacetylactis ATCC 13675 and mixtures thereof.

In another embodiment, the probiotic microorganism is selected from the group comprising: Streptococcus salivarus subspecies thermophilus type 1131, Enterococcus faecium SF68, Saccharomyces boulardii (Saccharomyces cerevisiae Hansen CBS 5296) and mixtures thereof.

In one embodiment, the probiotic microorganism is selected from the group comprising: Lactobacillus johnsonii, Lactobacillus casei, Bifidobacterium lactic, Lactobacillus paracasei, Lactobacillus acidophilus, Lactobacillus rhamnosus, Lactobacillus plantarum, Streptococcus thermophilus and mixtures thereof.

In one embodiment the probiotic microorganisms are bacteria. In another embodiment the bacteria are Lactobacillus species Bifidobacterium or mixtures thereof.

In one embodiment the bacteria are selected from the group comprising L. casei CRL431 (ATCC accession number 55344), L. casei ATCC 393, L. acidophilus ATCC 4356, L. rhamnosus ATCC 53103 and B. lactic BB12.

In one embodiment, the priobotic microorganism is L. casei 431.

Methods of preparing probiotic microorganisms are well known in the art. For example, cultures of probiotic bacteria can be purchased from a supplier, then can be suspended and grown in a suitable broth, such as MRS broth. The growth of the bacteria can be monitored by measuring the UV absorbance of the suspension.

The probiotic microorganisms for use in the invention can be prepared in any liquid growth medium suitable for microbial growth. Such media are known to persons skilled in the art. For example, the liquid growth medium might be LB broth or MRS broth. When the microorganisms are lactic acid bacteria, a whey or milk based growth medium such as cheese whey supplemented with minerals and nitrogen sources or skim milk is preferred.

### Suspension of Osmotically Shocked Microorganisms

In the method of the invention probiotic microorganisms are osmotically shocked before being combined with the particles comprising carbohydrates, protein and optionally fat. The terms “osmotic shock” and “osmotic stress” as used herein refer to a sudden change in the solute concentration around a cell, resulting in a rapid change in movement of water across the cell membrane. Under conditions of high concentrations of solute (for example salt), water is drawn out of the cells by osmosis. Methods of osmotically shocking microbial cells are well known in the art and any method can be used in the present invention.

In one embodiment, the probiotic microorganisms are osmotically shocked by increasing the osmolarity of a suspension of the probiotic microorganisms.

In one embodiment, the osmolarity is increased by increasing the solute concentration of the suspension of probiotic microorganisms.

Any form of edible salt may be used in the present invention to osmotically shock the probiotic microorganisms. Preferably salts such as sodium chloride, ammonium chloride, sodium bicarbonate, potassium chloride, sodium citrate, potassium citrate and potassium chloride are used.

Alternatively, the suspension of probiotic microorganisms can be osmotically shocked by the addition of other solutes, for example, mineral salts, sugar derivatives (for example lactose, glucose, sucrose or trehalose) or polyol compounds, for example glycerol or sorbitol.

In one embodiment, the solute concentration of the suspension of probiotic microorganisms is increased to about 0.2 to about 2M solute. In another embodiment, the solute concentration of the solute of probiotic microorganisms is increased to about 0.2 to about 1M of the solute.

In another embodiment, the solute concentration of the suspension of probiotic microorganisms is increased to about 0.4 to about 0.8M solute.

In one embodiment, sodium chloride is added to the suspension of probiotic microorganisms such that the suspension contains about 0.2 to about 1M sodium chloride, preferably about 0.4 to about 0.8M, more preferably about 0.6M sodium chloride. The suspension generally comprises the media in which the probiotic microorganisms were grown in.

In another embodiment, glucose is added to the suspension of probiotic microorganisms such that the suspension contains about 0.4 to about 2M glucose, preferably about 0.8 to about 1.6M, more preferably about 1.2M glucose.

To carry out the osmotic shock process, the media the probiotic cells are grown in may be supplemented with the solute, then growth of the cells continued. However, the cells may also be pelleted and then re-suspended in fresh media containing the osmotic shock agent.

As would be understood by a person skilled in the art, the suspension must contain a relatively high concentration of probiotic microorganisms before it is osmotically shocked.

Preferably a concentration of 0.2-1.5M of a salt, sugar derivative or polyol compound, is incorporated in the probiotic growth media to osmotically shock the culture. Concentrations ranging from between 0.4-0.8M, such as 0.6M, are preferred.

Preferably the suspension of probiotic microorganisms is not osmotically shocked until it has reached a concentration of 10⁶-10¹² cfu per ml of media, more preferably 10⁶-10⁹ cfu/ml. In one embodiment, the suspension is osmotically shocked when the probiotic microorganisms reach a concentration of 10⁶ cfu per ml of media. In some embodiments, the suspension is osmotically shocked when it reaches a concentration of 10⁷, 10⁸, 10⁹, 10¹⁰, 10¹¹ or 10¹² cfu per ml of media.

The process of osmotically shocking the probiotic microorganisms does not result in a significant decrease in viable cells. Accordingly, the concentration of the probiotic cells in the osmotically shocked suspension for use in process of the invention (the coating suspension) is preferably between 10⁶ to 10¹² cfu/ml. More preferably, the coating suspension contains 10⁸ to 10¹² cfu/ml. Most preferably the coating suspension contains 10¹¹ to 10¹² cfu/ml.

In one embodiment, the suspension of probiotic microorganisms has a pH of about 6 to 7.

In one embodiment, the suspension of probiotic microorganisms is osmotically shocked for a period between 2 and 10 hours. In another embodiment, the suspension of probiotic microorganisms is osmotically shocked for 4-8 hours.

In addition to osmotically shocking the probiotic cells, in some embodiments the cells are also heat shocked.

### Particles Comprising Carbohydrate, Protein and Optionally Fat for Use in the Process of the Invention

The particles for use in the process of the invention must comprise carbohydrate, protein and optionally fat.

The particles for use in the process of the invention generally comprise powders of consumable material.

In one embodiment, the particles comprise at least 3 wt % carbohydrate, 3 wt % protein and at least 3 we/0 fat.

In one embodiment, the particles comprise at least 5, 10, 15, 20, 25, 30, 35, 40, 45, 50, 55, 60, 65 or 70 wt % carbohydrate.

In one embodiment, the particles comprise at least 5, 10, 15, 20, 25, 30, 35, 40, 45, 50, 55, 60, 65 or 70 wt % protein.

In one embodiment, the particles comprise at least 5, 10, 15, 20, 25, 30, 35, 40, 45, 50, 55, 60, 65 or 70 wt % fat.

In one embodiment, the particles comprise 1-60% by weight fat, 5-80% by weight protein, 5-80% by weight carbohydrate, wherein the particles comprise amounts of fat, carbohydrate and protein that are less than or equal to 100%.

More preferably, the particles comprise 3-40% by weight fat, 10-50% by weight protein, 10-60% by weight carbohydrate wherein the particles comprise amounts of fat, carbohydrate and protein that are less than or equal to 100%. Most preferably, the particles comprise 20-35% by weight fat, 25-45% by weight carbohydrate and 15-30% by weight protein wherein the particles comprise amounts of fat, carbohydrate and protein that are less than or equal to 100%.

In one embodiment, the particles consist essentially of carbohydrate and protein. Small amounts of fat (<0.5 wt %) may be present.

In one embodiment, the particles to be coated are in the form of a powder. The powder may be a dairy powder. Alternatively, the powder to be coated may be soy based, for example, soy protein isolate or soy protein concentrate. Alternatively the powder to be coated may include in its composition a non-dairy creamer, maltodextrin, cocoa powder, malt powder or starch. Alternatively, the powder may comprise a blend of any of these ingredients.

In one embodiment, the particles comprise a milk powder. In one embodiment, the particles comprise skim milk powder.

In another embodiment, the particles comprise a non-dairy creamer.

In a further embodiment, the particles comprise a blend of milk powder and non-dairy creamer.

The particles may also include minerals, vitamins and other macromolecules, such as prebiotics, dietary fibres, peptides, free fatty acids, phytochemicals etc and a small percentage of moisture.

In one embodiment, the particles consist essentially of a dairy powder.

### Coating and Drying the Particles to Produce Consumable Coated Particles or Agglomeration Thereof Including Viable Probiotic Microorganisms

In the process of the invention a suspension of osmotically shocked probiotic microorganisms is used to at least partially coat particles comprising carbohydrates, protein and optionally fat. The coated particles may partially agglomerate. Agglomeration is the process by which smaller particles are bought together to form larger particles by sticking them together.

Where the particles are agglomerated, the osmotically shocked probiotic microorganisms both coat the particles, and are themselves surrounded by or coated with other particles. The coated particles or agglomeration of coated particles are then dried.

The process of the invention can be carried out using any methodology that results in the particles being at least partially coated with a suspension of osmotically shocked probiotic microorganisms. In preferred embodiments the coated particles are agglomerated.

Generally, the drying air introduced has a temperature in the range 20-65° C., preferably, 30-60° C. In one embodiment the process the present invention is carried out at a temperature of about 20-50° C. In another embodiment, the temperature is about 30-45° C., more preferably 30-35° C.

In one aspect, the invention provides a process for preparing consumable coated particles or an agglomeration thereof comprising:

a) at least partially coating particles comprising carbohydrate, protein and optionally fat with a suspension of osmotically shocked probiotic microorganisms; and
b) drying the coated particles or agglomeration thereof at between 20-50° C.
wherein the consumable coated particles or agglomeration thereof include viable probiotic microorganisms.

In one embodiment, the process is conducted using fluidised bed apparatus. There are three phases present in the fluidised bed process. The solid (base material), liquid (binding material) and gas (fluidising air), all of which interact with each other simultaneously. Fluidised beds promote high levels of contact between the phases. Fluidised bed processing involves introducing a pressurised fluid through a solid particulate substance under conditions that cause the solid/fluid mixture to behave as a fluid. In fluidised bed drying, the fluidisation gas supplies heat to evaporate the liquid phase.

In one embodiment, the process is carried out in a fluidised bed apparatus with an outlet temperature of 20-40° C., preferably 30-35° C. Alternatively the drying may be by other methods such as vacuum drying.

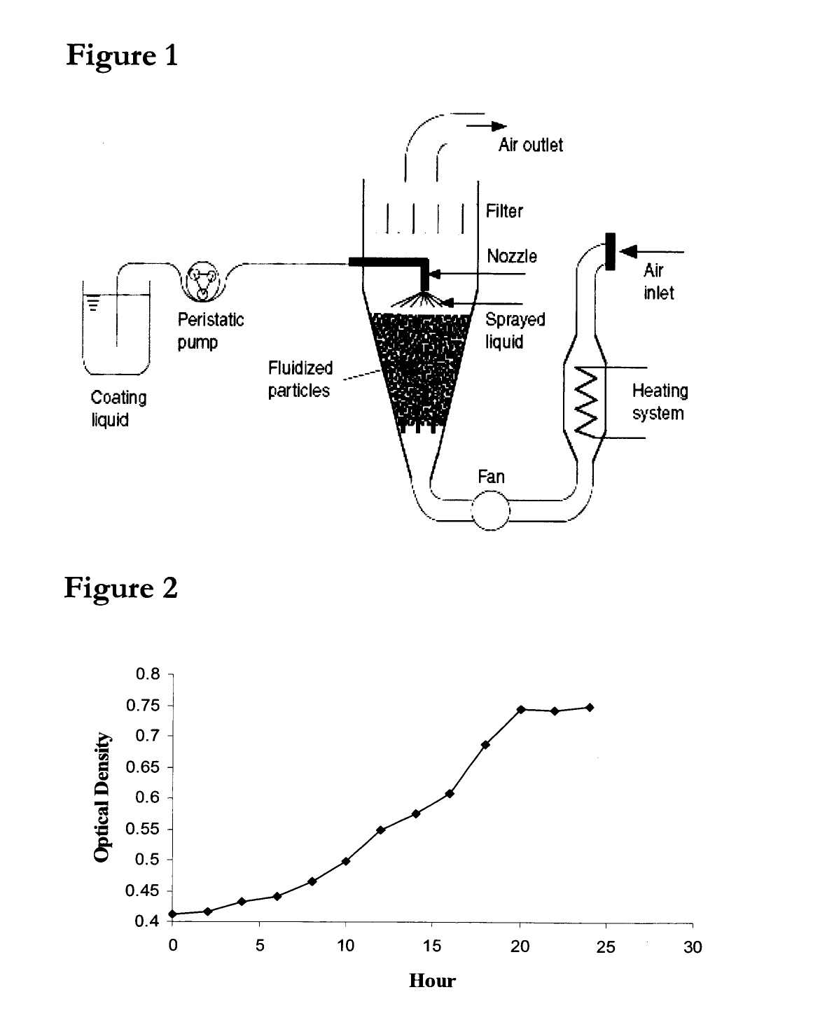

FIG. 1 illustrates a preferred process of the present invention where the solid base material (particles comprising carbohydrate, protein and optionally fat) is placed in an inverted truncated cone with a fine retention plate and an air distribution plate at its base. Warm air flows through the distribution plates and the embedding matrix become fluidised while a liquid comprising osmotically shocked probiotic cells is sprayed finely over the base material. Preferably, the humidity of the drying air is kept to the minimum practicable, such as 35 to 50% relative humidity.

After several cycles of wetting-drying the water portion of the spraying slurry causes agglomeration of the core materials. The probiotic cells become entrapped in agglomerated fine granules and finally the water is evaporated out due to the drying action. Free flowing, granulated powder mass is obtained with the same or lower level of water activity (A_{w}) as in the original base material.

Preferably, after the drying process is completed the consumable particles range from about 35 to 100 μm in diameter, more preferably 40-95 mm, most preferably, 50-80 μm. However, where particles agglomerate, diameters of up to 1 μm may be seen. When a fluidised bed apparatus is used, the particle size can be influenced by the atomisation air pressure, the liquid spray rate and the fluidisation air temperatures. When these are high, particles of lower diameter are obtained. Other influencing factors include the nozzle cone angle and proximity to the power bed. A person skilled in the art would be able to select the appropriate settings to produce particles of the desired size range. If required a particle size reduction step, for example, grinding or milling can be used.

The process of the invention provides an alternate to other methods of preserving probiotic microorganisms such as freeze drying, spray drying or encapsulation. The process of the invention has the advantage that it does not result in any significant loss in cell viability, and produces a product in which the probiotic microorganisms remain viable for long periods.

Accordingly, in one aspect, the invention provides a process for preparing consumable coated particles or an agglomeration thereof comprising:
a) at least partially coating particles comprising carbohydrate, protein and optionally fat with a suspension of osmotically shocked probiotic microorganisms; and
b) drying the coated particles or agglomeration thereof;
   wherein the consumable coated particles or agglomeration thereof include at least 10⁷ cfu/gram of viable probiotic microorganisms.

In another aspect the invention provides a process for preparing consumable coated particles or an agglomeration thereof comprising:
a) at least partially coating particles comprising carbohydrate, protein and optionally fat with a suspension of osmotically shocked probiotic microorganisms; and
b) drying the coated particles or agglomeration thereof and;
   wherein the consumable coated particles or agglomeration thereof include at least 10⁷ cfu/gram of viable probiotic microorganisms; and
   wherein the concentration of viable probiotic microorganisms decreases less than 2 log cfu/gram after 6 months storage at room temperature.

In the above aspects:

In one embodiment, the consumable coated particles or agglomeration thereof include at least 10⁸ cfu/gram of viable probiotic microorganisms.

In another embodiment the consumable coated particles or agglomeration thereof include at least 10⁹ cfu/gram of viable probiotic microorganisms.

In another embodiment the consumable coated particles or agglomeration thereof include at least 10¹⁰ cfu/gram of viable probiotic microorganisms.

In another embodiment the consumable coated particles or agglomeration thereof include at least 10¹¹ cfu/gram of viable probiotic microorganisms.

In one embodiment, the concentration of viable probiotic microorganisms decreases less than 1.8 log cfu/gram after 6 months storage at room temperature.

In one embodiment, the concentration of viable probiotic microorganisms decreases less than 1.6 log cfu/gram after 6 months storage at room temperature.

In one embodiment, the concentration of viable probiotic microorganisms decreases less than 1.4 log cfu/gram after 6 months storage at room temperature.

In one embodiment, the concentration of viable probiotic microorganisms decreases less than 1.2 log cfu/gram after 6 months storage at room temperature.

In one embodiment, the concentration of viable probiotic microorganisms decreases less than 1.0 log cfu/gram after 6 months storage at room temperature.

In one embodiment, the concentration of viable probiotic microorganisms decreases less than 0.8 log cfu/gram after 6 months storage at room temperature.

In another embodiment, the concentration of viable probiotic microorganisms decreases less than 0.6 log cfu/gram after 6 months storage at room temperature.

In another embodiment, the concentration of viable probiotic microorganisms decreases less than 0.4 log cfu/gram after 6 months storage at room temperature.

In another embodiment, the concentration of viable probiotic microorganisms decreases less than 0.2 log cfu/gram after 6 months storage at room temperature.

In one embodiment, the concentration of viable probiotic microorganisms decreases less than 2.0 log cfu/gram after 12 months storage at room temperature.

In one embodiment, the concentration of viable probiotic microorganisms decreases less than 1.8 log cfu/gram after 12 months storage at room temperature.

In another embodiment, the concentration of viable probiotic microorganisms decreases less than 1.6 log cfu/gram after 12 months storage at room temperature.

In one embodiment, the concentration of viable probiotic microorganisms decreases less than 1.4 log cfu/gram after 12 months storage at room temperature.

In another embodiment, the concentration of viable probiotic microorganisms decreases less than 1.2 log cfu/gram after 12 months storage at room temperature.

In another embodiment, the concentration of viable probiotic microorganisms decreases less than 1.0 log cfu/gram after 12 months storage at room temperature.

In another embodiment, the concentration of viable probiotic microorganisms decreases less than 0.8 log cfu/gram after 12 months storage at room temperature.

In another embodiment, steps a) and b) are carried out using fluidised bed apparatus.

In another embodiment, the coated particles or agglomeration thereof are dried at about 20-50° C.

The stability of the consumable particles or agglomeration thereof can be assessed by counting the cfu/gram after a period of storage at room temperature. Storage conditions can vary. While humidity control is not necessary, the samples should preferably be sealed in air-tight containers for maximum stability.

In one aspect the invention provides a process for preparing consumable coated particles or an agglomeration thereof including at least 10⁷ cfu/gram of viable L. casei 431 comprising:

a) at least partially coating particles comprising carbohydrate, protein and optionally fat with a suspension of osmotically shocked L. casei 431, cells and
b) drying the coated particles or agglomeration thereof;
wherein steps a) and b) are carried out using a fluidised bed apparatus; and
wherein the concentration of viable L. casei 431 cells decreases less than 1 log cfu/gram after 6 months storage at room temperature.

In one embodiment, steps a) and b) are carried out at 20 to 50° C.

In one embodiment, the concentration of viable L. casei cells decreases less than 2 log cfu/gram after 12 months storage at room temperature.

In one embodiment, the particles comprising carbohydrate, protein and optionally fat comprise a dairy powder.

The particles and particle agglomerations produced by the method of invention have application as ingredients in consumable food products. The unique processing conditions ensure that the probiotic microorganisms coated on and between the particles remain viable for long periods.

In one aspect, the invention provides consumable coated particles or an agglomeration thereof produced by a process of the invention.

In another aspect, the invention provides consumable coated particles comprising carbohydrate, protein and optionally fat, at least partially coated with osmotically shocked probiotic microorganisms, and/or an agglomeration of such particles.

In the above aspects:

In one embodiment, the consumable coated particles or agglomeration thereof include at least 10⁷, 10⁸, 10⁹, 10¹⁰ or 10¹¹ cfu/gram of viable probiotic microorganisms.

In another embodiment, the concentration of viable probiotic microorganisms decreases less than 2, 1.8, 1.6, 1.4, 1.2, 1.0, 0.8, 0.6, 0.4 or 0.2 cfu/gram after 6 months storage at room temperature.

In another embodiment, the concentration of viable probiotic microorganisms decreases less than 2, 1.8, 1.6, 1.4, 1.2, 1.0 or 0.8 cfu/gram after 12 months storage at room temperature.

In another embodiment the probiotic microorganism is L. casei 431.

### Consumable Food Products

Probiotic microorganisms are commonly found in fermented milk products such as yogurt. These products must be refrigerated and have short shelf lives. Producing cereal products containing large enough numbers of viable probiotic microorganisms to have health benefits is more difficult. Such products need to have long shelf lives—up to six months or a year—and the cell counts of viable probiotic microorganisms can fall to insignificant levels within a few days.

However, the particles and agglomerations of the invention retain high levels of viable probiotic microorganisms for several months, and therefore are ideal for use in consumable food products, particularly those products that need long shelf lives.

Accordingly, the invention also provides consumable food products comprising the consumable coated particles or agglomerations thereof of the invention.

To ensure that the viability of the probiotic cells is maintained for as long as possible in the consumable product, the probiotic containing particles, or agglomeration thereof, should be used as soon as possible. Preferably, the dried and coated particles will comprise around 10⁹-10¹⁰ cfu/gram. Preferably, food products will be fortified with 1-10% (w/w) of the coated particles produced by the method of the invention and the fortified food will then contain 10⁷-10⁹ cfu/gram.

More preferably, the food or ingredient produced by the method of the invention will contain 10⁷ to 10¹⁰ cfu/gram, most preferably 10⁷ to 10⁹ cfu/gram. However, lower numbers such as amounts of around 10³-10⁵ are also likely to provide beneficial effects. The particles comprising viable probiotic cells might be useful as a food product. For example, the dried particles might be a dairy powder ready for reconstitution and use as a substitute for fresh milk, for example.

The particles comprising viable probiotic microorganisns may be useful in dry foods or intermediate moisture food.

The availability of water in a food or beverage is referred to as water activity (A_{w}) of a food is determined by the amount of water in the food. The water activity influences the microbial growth and shelf stability of the final consumable product. A dry food is defined as having a value of A_{w}<0.25 while an intermediate moisture food is defined as having an A_{w} value of between 0.4-0.8. Pure water has a water activity of 10.0.

Examples of dry and intermediate moisture foods that the particles might be usefully incorporated into in the present invention are powders, dairy based beverages, malt/soy/cereal based beverages, breakfast cereal such as muesli flakes, fruit and vegetable juice powders, cereal and/or chocolate bars, confectionary, spreads, flours, milk or smoothies.

The consumable coated particles or agglomerates thereof are particularly suited for incorporation into or onto cereals. However, the consumable coated particles or agglomerates thereof may be consumed directly, after reconstitution in water to make a liquid beverage.

The amount of consumable particles or agglomeration thereof used varies with the consumable product into which it is to be incorporated.
For example, dairy products may be supplemented with up to 10% w/w of the consumable coated particles and agglomerates thereof. Cereals coated with the particles may comprise only 1 or 2% w/w.

Preferably, the probiotic microorganisms incorporated onto and into the particles and agglomeration thereof are viable at room temperature for between six months to one year. The gradual decrease in the concentration of viable probiotic microorganisms seen in the consumable particles or agglomeration thereof is reflected in the consumable food product. For example, a loss of about 1 log cfu/gram in the consumable particles or agglomeration thereof, will result in a loss of about 1 log cfu/gram in the consumable food product into which the former has been incorporated.

Such losses are much slower than losses generally seen in products containing probiotic microorganisms kept at room temperature. The losses are sufficiently low that even after several months, the consumable product can still deliver enough probiotic microorganisms to provide health benefits to the consumer.

The coated or agglomerated particles produced by the method of the invention may be used directly as food, or ingredients for foods, or food supplements, or nutraceuticals.

In one embodiment the food or food ingredient is a confectionary, milk, milk product, milk powder, reconstituted milk, cultured milk, yoghurt, drinking yoghurt, set yoghurt, drink, dairy drink, milk drink, food additive, drink additive, dietary supplement, nutritional product, medical food, nutraceutical or pharmaceutical.

These products may include any edible consumer product which is able to carry carbohydrate, protein or optionally fat or combinations thereof. Examples of suitable edible consumer products include aqueous products, baked goods, confectionary products including chocolate, gels, ice creams, cereals, reconstituted fruit products, snack bars, food bars, muesli bars, spreads, sauces, dips, dairy products including yoghurts and cheeses, drinks including dairy and non-dairy based drinks, milk, milk powders, sports supplements including dairy and non-dairy based sports supplements, fruit juice, food additives such as protein sprinkles and dietary supplement products including daily supplement tablets, capsules, soft gels and powders. Suitable nutraceutical compositions useful herein may be provided in similar forms.

Preferably, the particles produced by the method of the invention are a dried, semi-dried or intermediate dried food such as dairy or soy based powders, dairy based powdered beverages, malt/soy/cereal based beverages, breakfast cereal, fruit or vegetable juice powders. Alternatively, the particles produced by a method of the invention are used to fortify a semi-dried or intermediate dried food such as dairy or soy based nutritional powders, dairy based powdered beverages, malt/soy/cereal based beverages, breakfast cereal, fruit or vegetable juice powders, cereal bars, chocolate bars, confectionary, spreads, sauces or smoothies.

### Diseases to be Treated

Much study has been conducted on the effects of probiotic microorganisms in the prevention and treatment of disease.

Probiotics such as Lactobacillus rhamnosus GG and Bifidobacterium lactis BB-12 have been linked with preventing and treating infectious diarrhoea, particularly diarrhoea caused by rotaviruses in children (FAO/WHO, 2001).

Probiotics are also thought to be useful in restoring gut micro flora after an individual has been treated with antibiotics to prevent and/or treat an abnormal elevation in Clostridium difficile, which can result in diarrhoea (FAO/WHO, 2001).

Lactic acid bacteria have also been shown to be effective in vitro in inhibiting the growth of Helicobacter pylori, an organism known to cause type B. gastritis, peptic ulcers and gastric cancer (FAO/WHO, 2001).

Studies are also currently being conducted that support treating inflammatory and bowel syndromes such as pouchitis and Crohn's disease with probiotics.

Other research has linked probiotics with preventing some cancers and helping to alleviate constipation.

Researchers have also shown that probiotics can modify immune parameters such as activation of natural killer cells in the elderly, induction of mucous production, macrophage activation, the stimulation of sIgA and neutrophils at the site of probiotic action and stimulation of elevated peripheral immunoglobulins.

Other studies have shown evidence that probiotics are useful for treating cardiovascular disease, urogenital tract disorders, bacterial vaginosis; yeast vaginitis and urinary tract infections (FAO/WHO, 2001).

Therefore, treating individuals with probiotics has been shown to result in a range of health benefits. The particles and agglomerations thereof of the present invention may be used in pharmaceutical applications.

In one aspect, the invention comprises a pharmaceutical composition comprising the coated particles or agglomeration thereof, together with a pharmaceutically acceptable excipient.

Various aspects of the invention will now be illustrated in non-limiting ways by reference to the following examples.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### EXAMPLES

Examples 1-5 were carried out using the L. casei CRL431 strain.

### Example 1

### Identification of Growth Phases of L. casei CRL431 Strains

This experiment was designed to determine the beginning, midpoint and end point of log phase and the duration of stationary phase of the experimental strain L. casei CRL431.

Bacterial growth phases were identified by measuring the changes in optical density of the culture media (MRS broth) using a spectrophotometer (Hitachi, Inc.).

Freeze dried cells were rehydrated in MRS broth for 3 consecutive days and then inoculated in 10 ml MRS broth at 1.0% (v/v) and incubated at 37° C. Pre-sterilized peptone water in bottles (9 ml each) was also maintained at 37° C. for dilution purpose.

At each time point, 300 μl of the culture media was aseptically transferred into a 9 ml peptone water bottle. The culture media was diluted with 0.2% peptone water and mixed thoroughly using a vortex stirrer. The optical density of the diluted culture media was then measured at a wavelength of 610 nm. Readings were taken at 2 hour intervals for a total duration of 24 hours.

It was established that the mid log and early stationary phases for L. casei CRL431 were 14 and 20 hours from the initial inoculation time.

Results for this experiment showing the growth curve for L. casei CRL431 are shown in FIG. 2.

### Example 2

### Shock Treatments

### Osmotic Shock Treatment

L. casei CRL 431 cells were grown consecutively for three generations. Generally, one growth cycle of probiotic cells was completed in 17-20 hours. After this period an inoculum was inoculated into fresh growth media for growth of another generation. This process was repeated three times before the final cells were harvested.

After growth cycling for 3 consecutive days, bulk cultures were prepared with 2% (v/v) inoculation (4×400 ml MRS broth+4×8 ml inoculums) and incubated at 37° C.

Cells were incubated as described in Example 1 for 14 hours until mid log phase was reached. In each of two of the four 400 ml culture media, 100 ml of additional pre-sterilized MRS broth was added which contained 16 gm of sodium chloride. The salt concentration in the final 500 ml media was 0.6M.

The other two bottles of culture media were negative controls which were each supplemented with 100 ml of sterile MRS broth.

All four bottles were incubated for a further 6 hours until the cells reached their stationary growth phase (20 hours from inoculation). Cells were then harvested by centrifuging the four bottles at 7500 RPM for 10 minutes to obtain a cell pellet in each bottle. The cell pellets were then each re-suspended into 0.2% peptone water, washed by gentle shaking and further centrifuged to collect the washed cell pellets. Approximately 5 grams of cell pellet were obtained from each bottle.

### Heat Shock Treatment

MRS broth (2×400 ml) was inoculated with 2% (v/v) of revived culture stock and incubated at 37° C. for 20 hours.

The duran bottles containing the inoculated media were then placed in a circulating water bath pre-heated to 50° C. The bottles were held in the bath for 30 minutes with gentle shaking.

After 30 minutes, the media was immediately cooled down to room temperature by circulating tap water around the bottles. The cell harvesting, washing and pellet collection methods were the same as described above.

### Combined Heat and Osmotic Shock Treatment

The cell preparation and osmotic shock treatment were the same as described above. However, after growing for 20 hours the cells were washed and removed from the media containing sodium chloride, re-suspended into fresh sterile MRS broth and then subjected to heat shock according to the method described above.

### Example 3

### Embedding Using Fluidized Bed Drier

The cell growth conditions, osmotic shock, heat shock, combined stress and cell harvesting procedures were the same as set out in Examples 1 and 2.

The cell pellets obtained from centrifugation and subsequent washing were formed into a suspension by adding 0.2% sterile peptone water. For each sample, 12.50 grams of cell pellet obtained from 2 L of MRS broth was made up to 25 ml for spraying. A dry blend of whole milk powder (Fonterra Co-Operative, NZ) and commercial non-dairy creamer (Vanablanca35C from Kerry) was prepared to give a final composition as shown in Table 1.

TABLE 1

| | |
|---|---|
| Milk fat | 25.92% |
| Vegetable fat | 3.5% |
| Milk carbohydrate (lactose) | 34.20% |
| Glucose | 5.85% |
| Milk protein | 22.07% |
| Minerals | 5.14% |
| Moisture | 3.32% |

Two hundred and fifty grams of this blended powder was taken into the fluidized bed dryer (Glatt Inc. Germany). 25 mL of the L. casei cell slurry was intermittently sprayed onto the powder bed to ensure uniform and thorough dispersion of cells into the matrix. Fluidization and the drying process were running in parallel to the spraying of bacterial cell slurry.

The fluidizing air was maintained at 48-55° C. The samples were tested periodically for water activity (A_{w}) values and drying was continued until A_{w} reached below 0.25. The drying process took approximately 1 hour for each sample.

The dried samples were then packed in double layered low density polyethylene (LDPE) sachets and stored at 25° C. without humidity control or desiccated conditions to mimic the storage environment in commercial displaying shelves. Samples were periodically tested to obtain the cell count for viable Lactobacilli using a standard pour plate method.

To compare the results of this process with conventional freeze drying technology, a freeze dried L. casei sample was prepared by growing the cells in MRS broth and transferring into reconstituted skim milk followed by freeze drying. The dried culture was then dry blended with the same embedding matrix at 1% (w/w) as used in the fluid bed drying experiment (see Table 1) and shelf stability analysis was performed under similar storage conditions.

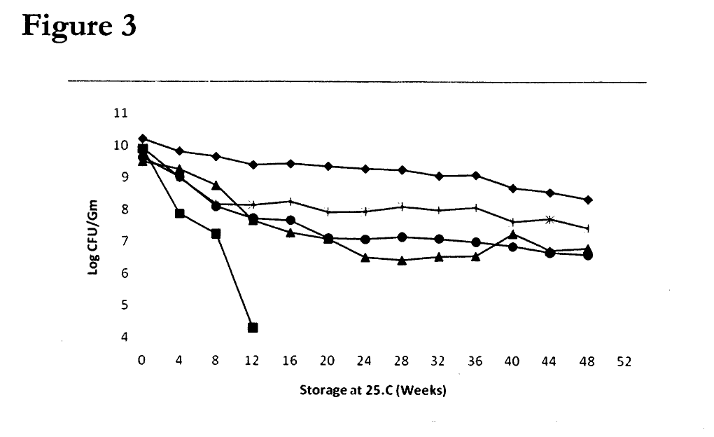

The results were duplicated and an average of these results is shown in logarithmic scale in FIG. 3 and discussed below.

### Results

The initial cell concentration was above 10⁹ cfu/gm level for each of the four samples. As shown in Table 2 below.

TABLE 2

| | |
|---|---|
| Combined treatment (osmotic and heat) | 10^{9.92} cfu/g |
| Heat stress | 10^{9.63} cfu/g |
| Osmotic | 10^{9.55} cfu/g |
| Control | 10^{9.50} cfu/g |

Storage at 25° C. with no humidity control or desiccated conditions, and moderate temperature caused loss in cell viability for all of the samples but the degree of cell death was different for each sample.

Maximum loss in viable cell count was observed in the commercial freeze dried sample where cell count was reduced to 4.30 log cfu/gm within 12 weeks. This level is well below the guidelines provided for by FAO/WHO (10⁶-10⁷/gm). Accordingly, further enumeration of this sample was discontinued after 12 weeks. The control and heat stressed samples showed a similar trend in cell mortality and at the end of 24 weeks the viable cell count for these samples was 6.6 and 7.0 log cfu/gm respectively. After 48 weeks the viable cell count was 6.81 and 6.60 log cfu/gm respectively. The effect of the combined stress treatment on cell viability was moderate (8.1 log cfu after 36 weeks and 7.45 log cfu after 48 weeks).

Cell mortality was most reduced in the L. casei cells that had undergone osmotic shock prior to the embedding process. This sample had a viable cell count of 8.43 log cfu/gm after storage for 36 weeks, which equates to a reduction in cell viability of just 1.12 log. After 48 weeks the sample showed a viable cell count of 7.68 log cfu/gm. The end result for this sample was well above the FAO/WHO guideline of delivering 6.0 log cfu of viable cells per gram in any food product.

Accordingly, this experiment demonstrates that the method of the present invention results in superior cell viability protection during long term storage in moderate temperature.

### Example 4

### Shelf Stability of Ingredient when Used in Commercial Foods

Because Example 3 demonstrated that the cells subjected to osmotic stress followed by drying were the most shelf stable, further stability testing in commercial product applications was carried out on a sample of these cells alone.

The shelf stable probiotic ingredient was prepared according to the same process described in Example 3. This ingredient was used to fortify commercially available intermediate moisture foods: a cereal bar (Cadbury Brunch Bar, Cadbury, NZ) and chocolate spread (Cottee's Chocolate Whizz, H J Heinz Co, Australia, Ltd) and tested for viable cell counts during the storage period.

The cereal bars were prepared by cutting the bar longitudinally in half. 1.0% (w/w) of the probiotic ingredient was then placed in the middle of the two halves. The cereal bars were re-packed in LDPE layered aluminium foils and heat sealed and were stored for 24 weeks at 25° C. with no humidity control.

6% (w/w) of the probiotic ingredient was mixed into commercially sought chocolate spread. The spread was then placed into sterile plastic containers with air tight caps. Samples were stored at 25° C. with no humidity control for 24 weeks.

### Results

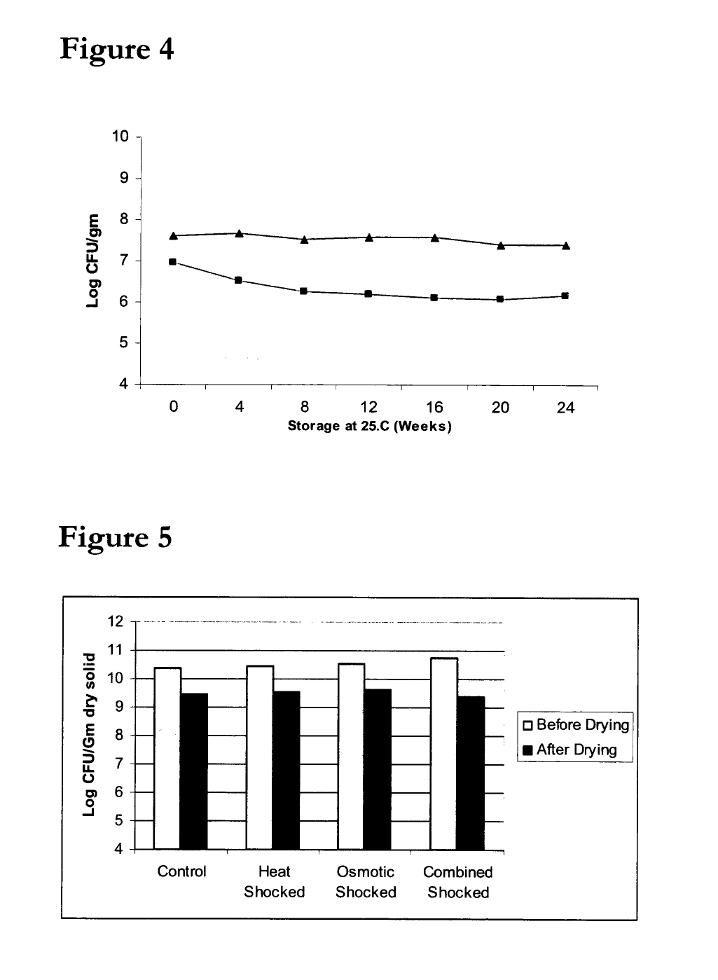

Results for this experiment are shown in FIG. 4 and discussed below.

The chocolate spread containing 6% (w/w) had an initial L. casei population of 7.63 log cfu/gm which remained almost steady during the 24 week storage period. At the end of the 24 week period the cell count had reduced to 7.41 log cfu/gm.

The cereal bars containing 1% (w/w) of the ingredient showed a lower initial count of 6.98 log cfu/gm and at the end of 24 weeks 6.2 log cfu/gm.

In both cases, the fortified products were able to maintain the minimum levels of probiotic suggested by FAO/WHO as a guideline.

There is scope for delivering a higher quantity of viable cells by increasing the initial cell load. Further research related to this is currently being undertaken.

### Example 5

### Microencapsulation in Sodium Alginate

### Cell Growth and Osmotic Shock

The cell growth conditions, osmotic shock and cell harvesting procedures were the same as set out in Examples 1 and 2.

### Microencapsulation

Four samples were prepared including a control, osmotically shocked cells, heat shocked cells and combined shocked L. casei cells. Each sample was then encapsulated as follows.

Sodium alginate solution (1% w/v) was autoclaved and 4 grams of centrifuged cell pellet was added to 100 gm of this solution and then homogenized in an Ultra-Turax mixer at 8500 RPM for 1 minute to uniformly disperse the cells. This core-wall mix was then sprayed over a sterile 4.0% CaCl₂ solution, using a 300 μm diameter nozzle, to form tiny gel particles. This is a standard extrusion technique for microencapsulating probiotic bacteria. The equipment used for encapsulation was an Inotech Encapsulator from EncapBioSystems AG, Greifensee, Switzerland.

The microcapsules were then hardened for 30 minutes under agitation (300 RPM). The CaCl₂ solution was decanted out and the beads were washed 2 times with sterile distilled water. The beads were then harvested by centrifuging at 4500 RPM for 3 minutes.

### Vacuum Drying Process

The centrifuged mass of alginate beads containing bacterial cells were subjected to a vacuum drying process. In order to impart minimum heat stress during the drying process, a temperature of 40 to 45° C. was maintained in the dryer by adjusting the vacuum level at −80 KPa. From an initial water activity level of ≈0.93-0.95, it took about 5 hours to dry the mass to an A_{w} level of 0.45-0.47.

### Lipid Film Coating Process

The coating material chosen was a mix of partially hydrogenated oils manufactured by Bunge India Pvt. Ltd., India (DALDA). This material is in solid form at ambient temperature and melts above 40° C. The fat was melted by placing the fat in a 50° C. water bath. Dried alginate capsules were mixed into the melted fat at 3:4 ratio. This ratio was chosen to add just enough oil for a proper visible coating. Higher quantity of oil was suspected to result in undesirable sensory quality and lesser payload of entrapped cells in the final ingredient. The applied coating was then solidified by cooling down the mix to the room temperature.

Uncoated and lipid coated alginate microcapsules were then dry blended with microcrystalline cellulose (MCC) (2 grams alginate capsules per every 5 grams of MCC). The blend was then packed in LDPE sachets and stored at 25° C.

Enumeration of Viable L. casei Cells

Alginate capsules (wet and dried) were dissolved into 0.2M sodium phosphate solution with the help of a stomachar to release the entrapped bacterial cells followed by serial dilution in 0.2% peptone water and pour plate counting on MRS agar.

### Results

The results of Example 5 are shown in FIGS. 5, 6 and 7 and are discussed below.

The initial cell counts in all four wet samples were above 10.00 log cfu/grams. This amount is considered satisfactory because when an end product contains 1% of these cells, a sufficient number of live probiotic cells (10⁸ cfu/grams) is delivered. However, after the drying process a considerable loss in viability was observed and the viable cell populations were declined to on average 9.50 log cfu/grams for all samples (FIG. 5). This level was still satisfactory if no further significant loss in viability occurred during the storage period.

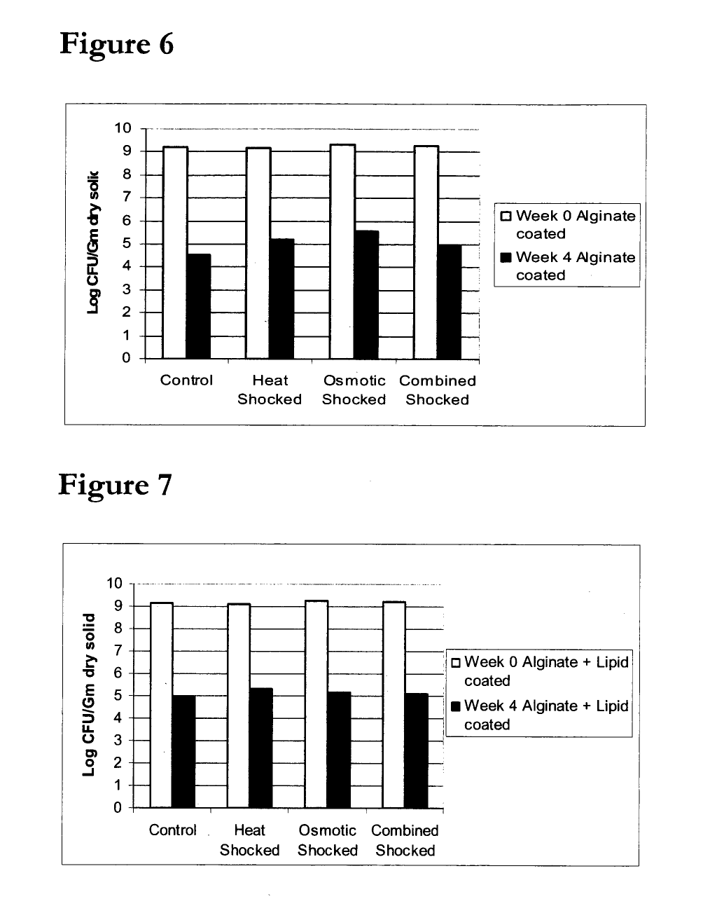

FIGS. 6 and 7 below show the decay in cell viability when the dried microcapsules (lipid coated and uncoated) were stored at 25° C. after mixing with MCC as an inert base material.

It is evident from FIGS. 5, 6 and 7 that all four samples have suffered a very high level of mortality due to the unfavourable storage conditions of a high storage temperature and low water activity. Stress adaptation in the cells only resulted in marginally improved viability compared to the control sample. No significant difference in mortality was observed in lipid coated samples.

Therefore, it was assumed that although applying such a barrier film has been reported to be effective for improving acid stability in the literature this might be useful only when the encapsulated probiotic is applied in a product matrix containing a high level of moisture.

As the viable cell count for all the samples decreased down below an acceptable level, further enumeration of the samples was discontinued.

As a parallel experiment for testing the shelf life stability in various product applications, the developed ingredient (dried/lipid coated alginate microcapsules) were fortified into cereal bars and chocolate spreads. However, an acceptable level of viability was not recorded after 4 weeks in any of the samples. Hence, the detailed results are not presented here.

### Example 6

### Stability of Different Strains of Bacteria when Subjected to the Process of the Invention

The techniques outlined in Examples 2 and 3 were used to produce samples of probiotic-coated particles using different probiotic strains. Four strains of bacteria were used to compare the stability of unstressed cells to osmotically stressed cells. The particles coated comprised the matrix described in Example 3 (Table 2). Coating and drying took place in a fluidized bed, as described in Example 3.

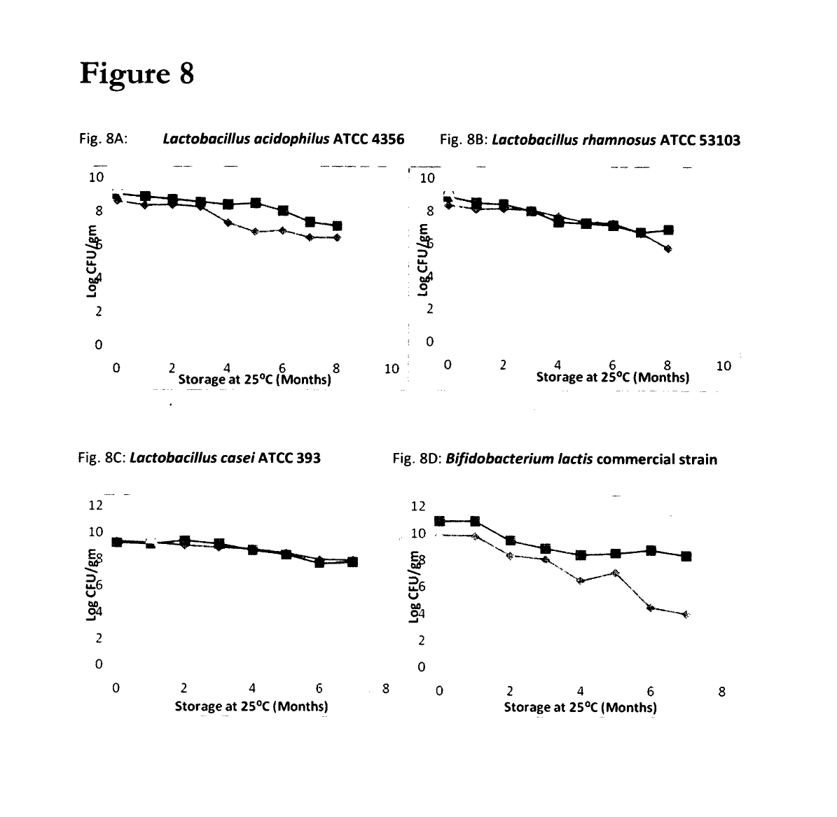

The results, shown in FIG. 8 demonstrate that significant improvement in storage viability was observed in osmotically stressed cells (▪) compared to unstressed cells (♦) in case of L. acidophilus ATCC 4356 (FIG. 8A) and Bifidobacterium lactic BB12 (FIG. 8D) strains and marginal improvements were recorded in case of L. casei ATCC 393 (FIG. 8C) and L. rhamnosus ATCC 53103 (FIG. 8B) strains. The freeze drying technique was used as a control ( ) where the residual viability of all of the bacteria was reduced to non-significant levels within 2 to 5 months.

### Example 7

### Comparison of the Method of the Invention with Conventional Drying Technology

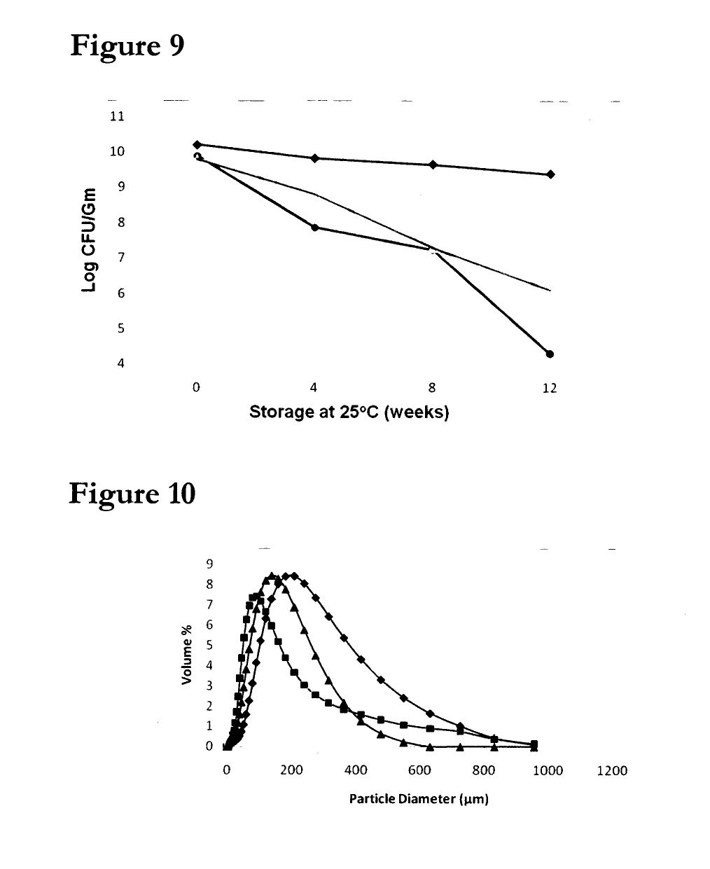

Osmoticially shocked L. casei cells prepared in accordance with Example 2 were used to coat the food matrix described in Example 3 (Table 1). Different techniques were used to coat and dry the particles. The results are shown in FIG. 9.

The combination of using osmotically shocked cells and coating and drying the particles in a fluidized bed provides a much more stable product than when the particles are coated with osmotically shocked cells and dried using alternative means, such as spray drying or freeze drying.

To understand the effect of low temperature fluid bed drying on the storage viability, L. casei cells were stabilised using standard spray and freeze drying techniques, keeping the level of osmotic stress and drying matrix same as that was used in case of the method of the invention. The results in FIG. 9 show that after 12 weeks of storage at 25° C. there was a very marginal decline in cell viability in the case of the fluidized bed drying whereas the spray and freeze dried cells were declined by about 3.0 and 3.5 log cfu/gm respectively.

It is recommended that the commercially available samples be stored at sub zero temperature. Therefore, these cells were the worst affected by ambient storage which was expected. The combination of osmotic stress and fluidised bed drying technology yielded superior results.

### Example 8

### Physical Characteristics of the Probiotic Ingredient Produced with the Method of the Invention

L. casei cells were osmotically stressed and encapsulated using the techniques and materials described in Examples 2 and 3. Different physical properties, e.g. particle size distribution, flow-ability, solubility were measured and compared with the two most common powders in the food industry, skim milk powder (SMP) and whole milk powder (WMP).

The particle size distribution of the probiotic powder of the invention was uniform (FIG. 10) with a surface-based mean diameter (D₃₂) of 125.6 μm which ranged from 7 to 832 μm. D₃₂ values for WMP and SMP were found to be 76.1 and 66.3 μm respectively. The diameter range for WMP was slightly shorter (6 to 550 μm) and SMP was very similar (5 to 954 μm). The significantly higher mean diameter clearly indicates the probiotic powder of the invention is highly agglomerated, which is a desirable property for easy handling and better dispersibility.

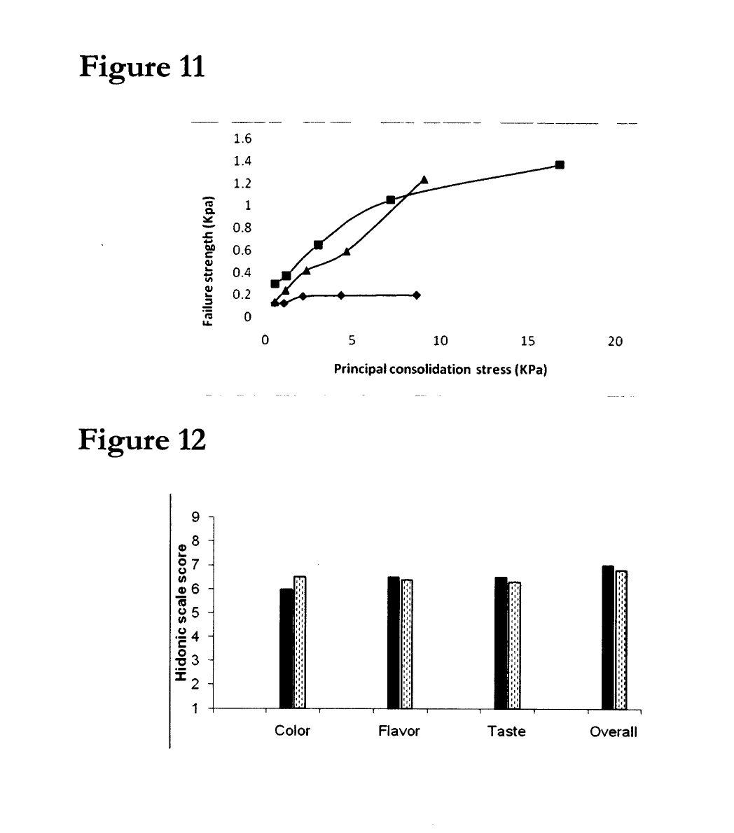

The flow function properties of the encapsulated probiotic powder of the invention, WMP and SMP are presented in FIG. 11. The flowability of a powder is defined by the slope of the curve. The highest flowability is recorded for SMP. The flow properties of WMP and probiotic powder were very close, with the probiotic powder being slightly better. The higher particle sizes due to agglomeration in the probiotic powder as reported above may be a probable reason for better flow properties. The average bulk densities of the samples were found to be 469.72 kg/m³, 540.52 kg/m³ and 583.25 kg/m³ for probiotic powder, WMP and SMP respectively. The comparatively lower density for the probiotic powder may also be due to the presence of highly agglomerated bigger sized particles as reported earlier (Szulc & Lenart, 2010).

The solubility index for probiotic powder, WMP and SMP were found to be 1.5, 1.0 and 1.0 ml respectively. The water activity of the probiotic powder was found to be 0.27 which is lower than both WMP (0.33) and SMP (0.30). A lower water activity means less free water available to the encapsulated bacteria which restricts their multiplication. Hence low water activity is considered to be a favorable factor for the ingredient's shelf stability.

### Example 9

### Sensorial Qualities of the Probiotic Powder of the Invention

The guideline issued by the FAO/WHO states that a probiotic rich product should be consumed in sufficient quantity so that at least 10 to 100 million live, viable cells are available in each day's diet. The malted milk beverage used for sensory evaluation was premixed with 10% of the probiotic powder. The probiotic ingredient of the invention was prepared by coating the particle matrix described in Example 3 (Table 1) with osmotically shocked L. casei CRL431 using a fluidised bed. The viable cell count of the whole mix was 4×10⁶ per gm. Therefore 10 gm of this beverage was found to be sufficient to meet daily requirements for an adult. Accordingly, the beverage was reconstituted by adding 90 ml of pasteurized milk (3.5% fat) preheated at 65° C. Immediately after preparation of the drink, it was served in 40 ml white plastic cups to a panel of 10 informed sensory tasters.

The experimental protocol followed was similar to the work reported by Guergoletto et. al. 2009. A triangular test was performed to spot any difference between the reconstituted malted beverage with or without the probiotic powder of the invention added into it. The acceptance levels of the prepared drink in terms of flavor color, taste and overall acceptance of the probiotic powder fortified drink was compared with a control sample with no probiotics and 10 tasters' opinions were evaluated. The samples were presented sequentially and a structured nine-point hedonic scale was used for recording the judgements. The scale is designed to quantify the qualitative opinions of the tasters in a range of one to nine score with one being ‘disliked very much’ and nine being ‘liked very much’.

In the organoleptic quality tests the overall acceptance score in a scale of 1 to 9 was calculated by averaging the scores given by 10 tasters. For the malted milk beverage with no added probiotic ingredient the score was 7.0. For the probiotic drink it was 6.8, which indicates almost equal acceptance for both (FIG. 12). The flavor and taste profiles of both drinks were also very similar with a marginal difference in the color between the two drinks (FIG. 12). In the triangular test, 7 out of 10 judges could not identify the difference between samples with and without added probiotic ingredient. Overall, no adverse comment regarding the acceptability of the particular samples with added probiotics was received.

### Example 10

### Heat Stability of the Encapsulated Probiotic Cells of the Invention in Simulated Powdered Beverage Drinking Conditions

Application of the probiotic powder of the invention in shelf stable dry nutritional powder was tested by dry blending 10 wt % with a malted beverage powder containing malt extract, cocoa, sugar, milk solids, caramel and glucose procured from the local market. An important criterion for successful delivery of live probiotic bacteria in a malted beverage is the heat stability of the strain because it is a common practice and also instructed by the manufacturer to consume after adding hot but not boiling water. Therefore, in this trial similar drinking condition were stimulated by directly adding 90 ml of hot water at 55° C., 65° C., and 75° C. to 10 gm of the malted beverage+probiotic ingredient of the invention. The live cell counts were enumerated after 5 and 30 minutes. The 30 min time period was observed to be the maximum time necessary for the drink to reach room temperature from the maximum hot water (75° C.) addition point and presumably the maximum time one would need to finish a cup of beverage.

The results (Table 3) showed that when 90 ml of water at 75° C. was added to 10 gm of the blended beverage (malted beverage+probiotic ingredient) and within 5 min the mix was tested for viable count, there was a decline of 0.5 log from the original cell population of the mix (8.4 to 7.9 log cfu/gm). After 30 min of holding, the temperature reduced to 35° C. and the cell population came down further to 7.3 log cfu/gm. If we assume that the drink was consumed over a long period (30 min), the recommended level of viable probiotic bacteria was still maintained in it. In case of adding water at 65° C., the cell population remained almost the same after 5 min and came down slightly to 7.9 log cfu/gm after 30 min when the temperature of the drink was recorded as 25° C.

Water added at 55° C. did not have any impact on viable cell count at all. Therefore it can be seen that though the recommended drink preparation technique involved exposure of the bacterial cells to elevated temperatures, even after considering the probable loss in viability, a sufficient amount of live cells to meet the recommended levels was still available to the consumer.

TABLE 3
Effect of hot water addition at different temperatures
on L. casei CRL431 cell viability when
encapsulated in the probiotic ingredient of the invention.

| | Original | | | |
|---|---|---|---|---|
| | cell | After | After | Final mix |
| Added | population | 5 Min | 30 Min | temperature |
| water | (Log | (Log | (Log | (° C.) |
| temperature | cfu/gm) | cfu/gm) | cfu/gm) | after 30 min |
| At 75° C. | 8.4 | 7.9 | 7.3 | 35 |
| At 65° C. | 8.4 | 8.3 | 7.9 | 25 |
| At 55° C. | 8.4 | 8.5 | 8.4 | 25 |

### Example 11

### Comparison of Storage Viability of Probiotic Ingredient of the Invention with Probiotic Ingredients Produced Using Alternative Matrices

A probiotic ingredient of the invention was produced using osmotically shocked L. casei CRL431 cells prepared in accordance with Example 2. The cells were used to at least partially coat particles of carbohydrate, protein and optionally fat using a fluidised bed, as set out in Example 3.

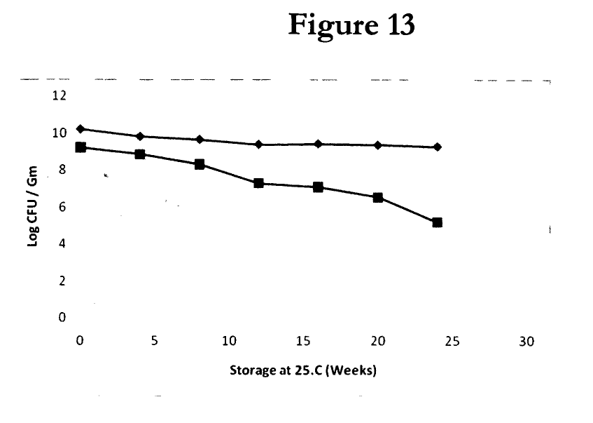

Comparative examples were also produced by at least partially coating particles of (a) milk protein isolate and (b) glucose, in a fluidised bed with L. casei cells prepared in the same way. The results are shown in FIG. 13.

The experiment shows that all three components of the particle matrix (ie, carbohydrate, optionally fat and protein) are necessary to achieve the superior stability properties demonstrated by the probiotic ingredient of the invention.

Even when the L. casei are osmotically shocked prior to the coating step, they do not remain viable when coated onto particles of glucose or protein only.

### INDUSTRIAL APPLICATION

The present invention has utility in producing probiotic microorganisms that retain viability when stored at room temperature for long periods of time. Accordingly, the process of the present invention produces probiotic microorganisms that can be incorporated in shelf stable dry and intermediate moisture foods.

Those persons skilled in the art will understand that the above description is provided by way of illustration only and that the invention is not limited thereto.

### REFERENCES

Adamiec, J. (2009). Drying of Microorganisms for Food Applications. In C. Ratti (Ed.), Advances in Food Dehydration: CRC Press.
Anal, A. K., & Singh, H. (2007). Recent advances in microencapsulation of probiotics for industrial applications and targeted delivery. Trends in Food Science & Technology, 18(5), 240-251.
FAO/WHO (2001). Report of a Joint FAO/WHO Expert Consultation on Evaluation of Health and Nutritional Properties of Probiotics in Food Including Powder Milk with Live Lactic Acid Bacteria. Cordoba, Argentina.
Guergoletto, K. B., Magnani, M., Martin, J. S., Andrade, C., & Garcia, S. (2010) Survival of Lactobacillus casei (LC-1) adhered to prebiotic vegetal fibers. Innovative Food Science & Emerging Technologies, 11(2), 415-421.
Lee, Y. K., & Salminen, S. (2009). Handbook of Probiotics and Prebiotics (2nd Edition ed.): Wiley
Meng, X. C., Stanton, C., Fitzgerald, G. F., Daly, C., & Ross, R. P. (2008). Anhydrobiotics: The challenges of drying probiotic cultures. Food Chemistry, 106(4), 1406-1416.
Ubbink, J., & Kruger, J. (2006). Physical approaches for the delivery of active ingredients in foods. Trends in Food Science and Technology, 17(5), 244-254.

1. A process for preparing consumable coated particles or an agglomeration thereof comprising:
a) at least partially coating (i) particles comprising carbohydrate and protein, or (ii) particles comprising carbohydrate, protein and fat, with a suspension of osmotically shocked probiotic microorganisms; and
b) drying the coated particles or agglomeration thereof;
wherein the consumable coated particles or agglomeration thereof include viable probiotic microorganisms.

2. The process according to claim 1 wherein the probiotic microorganisms are selected from L. casei CRL431, L. casei ATCC 393, L. acidophilus ATCC 4356, L. rhamnosus ATCC 53103 and B. lactis BB12.

3. The process according to claim 1 wherein the probiotic microorganisms were osmotically shocked using NaCl.

4. The process according to claim 1 wherein the particles of carbohydrate and protein, or the particles of carbohydrate, protein and fat, comprise a milk powder.

5. The process according to claim 1 wherein steps a) and b) are carried out using a fluidised bed apparatus.

6. The process according to claim 1 wherein the dried coated particles or agglomeration thereof includes at least 10¹⁰ cfu/gram of viable probiotic microorganisms.

7. The process according to claim 1 wherein the viable probiotic microorganisms are present at a concentration that decreases less than 2.0 log cfu/gram after 6 months storage at room temperature.

8. The process according to claim 1 wherein the viable probiotic microorganisms are present at a concentration that decreases less than 2.0 log cfu/gram after 12 months storage at room temperature.

9. The process according to claim 1 wherein step b) is carried out at about 20-50° C.

10. A process for preparing consumable coated particles or an agglomeration thereof including viable L. casei 431 at a concentration of at least 10⁷ cfu/gram, comprising:
a) at least partially coating (i) particles comprising carbohydrate and protein, or (ii) particles comprising carbohydrate, protein and fat, with a suspension of osmotically shocked L. casei 431 cells; and
b) drying the coated particles or agglomeration thereof,
wherein steps a) and b) are carried out using a fluidised bed apparatus, and
wherein the concentration of viable L. casei 431 cells decreases less than 1 log cfu/gram after 6 months storage at room temperature.

11. Coated particles or an agglomeration thereof produced by the process of claim 1.

12. A composition one or more of (i) consumable particles comprising carbohydrate and protein, said particles being coated with osmotically shocked probiotic microorganisms, (ii) consumable particles comprising carbohydrate° protein and fat, said particles being coated with osmotically shocked probiotic microorganisms, and (iii) an agglomeration of the particles of either or both of (i) and (ii).

13. The composition of claim 12 which comprises viable probiotic microorganisms
at a concentration that decreases less than 2.0 log cfu/gram after 6 months storage at room temperature.

14. The composition of claim 12 which comprises viable probiotic microorganisms at a concentration that decreases less than 2.0 log cfu/gram after 12 months storage at room temperature.

15. A consumable product comprising the composition of claim 12.

16. The consumable product according to claim 15 which is a confectionary, milk, milk product, milk powder, reconstituted milk, cultured milk, yoghurt, drinking yoghurt, set yoghurt, drink, dairy drink, milk drink, food additive, drink additive, cereal, dietary supplement, nutritional product, medical food, nutraceutical or pharmaceutical.

17. A pharmaceutical composition comprising the composition of claim 12; and a pharmaceutically acceptable excipient.
